(19) United States
(12) Patent Application Publication
(10) Pub. No.: US 2023/0270910 A1
(43) Pub. Date: Aug. 31, 2023

(54) Apparatus for dispensing a volatile substance, in particular an aroma or insectide, into ambient air

(71) Applicant: SWIF GmbH South West lnternational Fragrance, Kaiserslautern (DE)

(72) Inventor: Jörn Karg, Kaiserslautern (DE)

(21) Appl. No.: 18/019,265

(22) PCT Filed: Aug. 6, 2021
(86) PCT No.: PCT/EP2021/072010

§ 371 Date: Feb. 2, 2023

(30) Foreign Application Priority Data
Aug. 7, 2020 (DE) 10 2020 120 951.7

(51) Int. Cl. A61L 9/12

(52) U.S. Cl. CPC A61L 9/12; A61L 2209/15; A61L 2209/13

(57) ABSTRACT

Apparatus for releasing a volatile substance, in particular a fragrance or an insecticide, into ambient air, the apparatus includes a housing made of a first material which has a plant-based fiber material, and a barrier layer having a barrier function against permeation of the volatile substance, and arranged inside the housing, the apparatus includes an insert made of a second material, the second material is provided or impregnated with the volatile substance, wherein the second material has one or more of: i) a plant-based fiber material or ii) sucrose or starch or iii) a mineral material.

### PRIORITY

This application is a US National Stage entry of PCT/EP2021/072010 filed on Aug. 6, 2021, which claims priority to DE 10 2020 120 951.7 filed on Aug. 7, 2020, all of which are hereby incorporated by reference herein for all purposes.

### FIELD

The present invention relates to an apparatus for releasing a volatile substance, in particular a fragrance or an insecticide, into the ambient air, having a housing made of a first material which has a plant-based fiber material, in particular paper, cardboard, or paperboard.

### BACKGROUND

Such apparatus are known, for example, from U.S. Pat. No. 4,258,874 A, 4,166,565A, and 4,279,373 A, and have a housing embodied, for example, from paper or cardboard. A releasing device is typically arranged inside the housing and can release a volatile substance, e.g., a fragrance, during use of the apparatus. It has proven disadvantageous with these apparatuses that the volatile substance can diffuse through the housing made of paper or paperboard even prior to the apparatus actually being used. Because of this, only a reduced quantity of the volatile substance is available to the user during the usage interval. A further drawback is that residues that are not biodegradable typically remain in the housing after the apparatus has been used. This complicates disposal and recycling of such apparatus.

A further apparatus for releasing a fragrance is known from U.S. Pat. No. 4,712,737 A. This apparatus comprises a housing made of coated cardboard and a releasing device arranged inside the housing that can be formed, for instance, from a gel system, woven material, or nonwoven material. In this apparatus, as well, residues that are not biodegradable may remain in the housing after use.

### SUMMARY

The object of the present invention is to facilitate the disposal and/or recycling of an apparatus for releasing a volatile substance into the ambient air.

For attaining the object, an apparatus for releasing a volatile substance, in particular a fragrance or an insecticide, into the ambient air is suggested,
having a housing made of a first material which has a plant-based fiber material, in particular paper, cardboard, or paperboard, and a barrier layer with a barrier function against the permeation of the volatile substance, and,
having, arranged inside the housing, an insert made of a second material, wherein the second material is provided, in particular impregnated, with the volatile substance, wherein the second material is one of the following materials or has one or a plurality of the specified materials:
   a plant-based fiber material or
   sucrose or starch or
   a mineral material.

Such apparatus can also be called air fresheners or insecticides or insect repellants. The inventive apparatus comprises a housing made of a first material. This first material comprises a plant-based fiber material, for example paper, cardboard, or paperboard, and can therefore be easily disposed of or recycled after use. A further component of the apparatus is an insert made of a second material that differs from the first material. Thus, the first and second materials can be selected specifically for their individual function. This is because while the first material of the housing must provide a barrier function for the volatile substance prior to the apparatus being used, the second material should have the best possible properties for releasing the volatile substance. The second material is a plant-based fiber or sucrose or starch or a mineral material. The second material is provided, in particular impregnated, with the volatile substance, so that the latter can be released from the housing into the ambient air. The insert of the apparatus should also be simple to dispose of or recycle after use. It is possible to dispose of the housing and the insert together, i.e., without having to separate them, e.g., with waste paper or packaging waste (“yellow bag”).

According to the invention it is provided that the first material has a barrier layer with a barrier function against permeation of the volatile substance. Such a barrier layer shall be understood to be a layer having low permeability for the volatile substance. The barrier layer can prevent the volatile substance from escaping from the housing prior to the apparatus being activated. Due to the barrier layer, high enough partial pressure of the volatile substance can be maintained in the interior of the housing for further diffusing of the volatile substance out of the insert to be prevented.

The barrier layer preferably comprises polyamide, polyester, aluminum, ethylene vinyl alcohol, silicon dioxide, or polyvinyl alcohol. These materials have good barrier properties, in particular low permeability for volatile substances, in particular fragrances and insecticides. One embodiment with polyvinyl alcohol offers the advantage that the barrier layer is water soluble and is therefore simple to dispose of. Overall simplified recycling of the apparatus is thus made possible.

The first material can comprise a plurality of barrier layers. For example, it can be provided that the barrier layers have different permeabilities for different volatile substances. In this way a housing can be obtained that has a barrier function for a plurality of volatile substances, for example, for a plurality of substances in a mixture of substances, e.g., a fragrance mixture.

It is preferred when the barrier layer is arranged in contact with a ply made of the plant-based fiber material. In this way a compact embodiment of the first material can be obtained. Alternatively, a further layer, for example a protective layer or a sealing layer, can be arranged between the barrier layer and the ply made of the plant-based fiber material.

The barrier layer is preferably arranged between two plys made of the plant-based fiber material. Such an embodiment offers the advantage that both the interior and the exterior have the visual appearance of the plant-like fiber material. Furthermore, the barrier layer can be protected from environmental factors by the two plys made of the plant-based fiber material. When using a barrier layer made of polyvinyl alcohol, the former can be protected from the effects of water, for example, by the two plys, and thus undesired damage to the barrier layer can be prevented. Optionally, a further layer, for example a protective layer or a sealing layer, can be arranged between the barrier layer and the plys made of the plant-based fiber material.

Alternatively, it is preferred when the barrier layer is embodied as a coating of a ply made of the plant-based fiber material, in particular as a coating on an inner side of the housing. It is particularly preferred when the first material has exactly one ply of the plant-based fiber material coated on the inner side with the barrier layer.

One advantageous embodiment of the invention provides that the housing has a stand surface on which the housing can stand on a subsurface, wherein the stand surface is water repellant. For example, the housing can have a water-repellant coating in the region of the stand surface. The coating can comprise a nanomaterial.

One advantageous embodiment of the invention provides that the plant-based fiber material is paper or cardboard or paperboard, for example, mechanical pulp board or wood pulp.

One advantageous embodiment of the invention provides that the plant-based material is mechanical pulp, in particular ground wood pulp, or pulp or cotton fiber or grass fiber.

One advantageous embodiment of the invention provides that the mineral material is rock flour or bentonite or silica gel.

One advantageous embodiment of the invention provides that the insert has a plurality of plys of the second material connected to one another, in particular by an adhesive, in particular a plurality of plys of mechanical pulp board. Because the insert is formed from a plurality of plys of the plant-based fiber material, the weight of the insert, and thus also the weight of the entire apparatus, can be adjusted. It has been found that the users of such apparatus find it more valuable when the apparatus have a certain weight—that is, are not too light in weight. Therefore, a prespecified weight of the apparatus can be set by selecting the number of plys, for example four or five or six or seven or eight. In addition, as a result of the increased weight of the insert, there is the advantage that the volatile substance, in particular the fragrance or the insecticide, is distributed over a larger mass of the insert. Because of this, the volatile substance is diluted such that no unpleasantly strong odor is perceivable in the immediate vicinity of the housing of the apparatus. This circumstance can also contribute to the apparatus being perceived by the user as more valuable. As an alternative to the plys being connected using an adhesive, the plys can be connected using one or a plurality of nails, clamps, eyelets, bolts, or strips, for example a strip made of metal, wood, a polymer, glass, or stone.

If the insert has a plurality of plys of mechanical pulp board, it is advantageous when the mechanical pulp board comprises a layer of ground wood pulp arranged between two cover layers. The cover layers preferably each comprise pulp or paper, particularly preferably pulp and paper. The thickness of the mechanical pulp board is preferably in the range of 0.5 mm to 2.5 mm, for example 0.8 mm, 0.9 mm, 1.2 mm, 1.4 mm, 1.6 mm, 1.8 mm, 2.0 mm, or 2.4 mm.

According to one advantageous embodiment it is provided that the plys each have an area density in the range of 350 g/m² to 950 g/m², preferably 450 g/m² to 600 g/m², or 600 g/m² to 800 g/m². Plys having such an area density have proven to be particularly good for adjusting the weight of the insert.

Alternatively, it is advantageous when each of the plys has a thickness in the range of 0.2 mm to 2.5 mm and an area density in the range of 350 g/m² to 950 g/m², preferably:
a thickness in the range of 0.8 mm to 1.0 mm and an area density in the range of 350 g/m² to 400 g/m², or,
a thickness in the range of 1.1 mm to 1.3 mm and an area density in the range of 450 g/m² to 550 g/m², or,
a thickness in the range of 1.3 mm to 1.5 mm and an area density in the range of 490 g/m² to 570 g/m², or,
a thickness in the range of 1.5 mm to 1.7 mm and an area density in the range of 580 g/m² to 660 g/m², or,
a thickness in the range of 1.7 mm to 1.9 mm and an area density in the range of 630 g/m² to 750 g/m², or,
a thickness in the range of 1.9 mm to 2.1 mm and an area density in the range of 700 g/m² to 850 g/m², or,
a thickness in the range of 2.3 mm to 2.5 mm and an area density in the range of 800 g/m² to 1000 g/m².

Plys having such a combination of thickness and area density have proven to be particularly good for adjusting the weight of the insert.

According to one advantageous embodiment of the invention, it is provided that the adhesive is biodegradable. By using a biodegradable adhesive it is possible to provide an insert that is simpler to dispose of, in particular that is simpler to recycle or subject to industrial composting. It is particularly preferred that the adhesive conforms to European Standard EN 13432 and is “completely biodegradable” according thereto. Alternatively, an adhesive that is not biodegradable can be used.

One advantageous embodiment of the invention provides that the insert has at least a first ply, embodied in particular with the full surface area, and a at least one second ply having at least one recess, in particular a punched hole. The punched hole can form a receiving element for the volatile material. In this way it is possible to add the volatile material, for example in liquid form, to the receiving element formed by the recess. The volatile material can collect in the insert starting from the recess. The at least one recess can be embodied longitudinally, for example as a groove.

In this context, an embodiment in which the second ply forms an outer surface of the insert has proven advantageous.

One advantageous embodiment provides that the insert has a plurality of second plys, wherein the recesses of the plurality of second plys are arranged congruently above one another.

One advantageous embodiment of the invention provides that a time-temperature indicator is arranged on an outer side of the housing. The end of the useful life of the apparatus can be displayed by the time-temperature indicator. In this way the apparatus can display to a user whether the apparatus can still be used or can be disposed of or provided for recycling. The time-temperature indicator preferably comprises heat and UV light-sensitive pigments. These pigments can be activated by a manufacturer of the apparatus at the end of the production process by irradiating with UV light, wherein the UV dosage used here conforms to the shelf life. It is particularly preferred that the heat and UV light-sensitive pigments are covered by a film that is impermeable to UV light so that later manipulation using UV light is not possible.

One advantageous embodiment of the invention provides that the housing has a first housing part and a second housing part, wherein the first housing part and/or the second housing part comprises a receiving element for the insert, wherein the first housing part comprises one or a plurality of openings, in particular through-holes, through which the volatile substance can be released into the ambient air, and the second housing part can be displaced from a closed position, in which it covers the openings, in particular through-holes, of the first housing part to an open position, in which the openings, in particular through-holes, of the first housing part are exposed for releasing the volatile substance. In one such embodiment, the release of the volatile substance can be activated by displacing the two housing parts relative to one another. In the closed position, the second, in particular outer, housing part covers the through-holes in the first, in particular inner, housing part. Since the first material of the inner and outer housing part limit, in particular prevent, escape of the volatile substance, the partial pressure of the volatile substance within the housing remains constant at a level preventing escape of the volatile substance from the insert. When the apparatus is activated by displacing the housing parts, the one or plurality of through-holes are exposed. Consequently, ambient air can circulate through the through-holes and the volatile substance can be released into the ambient air.

The first and second housing parts preferably have a basic outline of identical shape, for example round, triangular, rectangular, in particular square, pentagonal, hexagonal, or octagonal.

A locking element that prevents complete separation of the two housing parts from one another is preferably arranged on the first housing part and/or second housing part. Because of this, undesired exposure of the insert, in particular by children, can be prevented. In this way it is possible to increase child safety using such an embodiment.

One alternative advantageous embodiment of the invention provides that the housing has a first housing part, a second housing part, and a third housing part, wherein the first housing part and/or the second housing part comprises a receiving element for the insert, wherein the third housing part has one or a plurality of through-holes through which the volatile substance can be released into the ambient air, wherein the first housing part and/or the second housing part can be displaced from a closed position, in which the first housing part and/or the second housing part covers the through-holes of the third housing part, to an open position, in which the through-holes of the third housing part are exposed for releasing the volatile substance. In such an embodiment, the volatile substance can be released by displacing the first and/or second housing part relative to one another to the third housing part. In the closed position, the first and/or second housing part covers the through-holes in the third housing part, which is arranged in the closed position preferably within the first and/or second housing part. Since the first material of the housing limit, in particular prevent, escape of the volatile substance, the partial pressure of the volatile substance within the housing remains constant at a level preventing escape of the volatile substance from the insert. When the apparatus is activated by displacing the first and/or second housing part relative to the third housing part, the one or plurality of through-holes are exposed. Consequently, ambient air can circulate through the through-holes and the volatile substance can be released into the ambient air.

In one such embodiment, the first housing part and the second housing part can be embodied identical. The third housing part can have the shape of a tube, wherein the basic outline of the tube is of the identical shape as a basic outline of the first and/or second housing part, in particular round, triangular, rectangular, in particular square, pentagonal, hexagonal, or octagonal.

A first locking element is preferably arranged on the third housing part and cooperates with a further first locking element on the first housing part and a second locking element is arranged on the third housing part and cooperates with a further second locking element on the second housing part. Complete separation of the first and second housing parts from the third housing part can be prevented using these locking elements. Because of this, undesired exposure of the insert, in particular by children, can be prevented. Thus, child safety can be increased using such an embodiment.

According to one advantageous embodiment, a gap between the first housing part and the second housing part in the closed position is covered by a removable barrier element having or embodied by a barrier material having a barrier function against permeation of the volatile substance. The barrier material can be, for example, polyamide, polyester, aluminum, or polyvinyl alcohol. The removable barrier element is preferably embodied as a sleeve. Alternatively, it can be provided that the removable barrier element is embodied as a release film.

In one apparatus according to one of the embodiments described in the foregoing having a first and a second and possibly a third housing part, it is preferably provided that the second housing part, starting from the closed position, is movable in the direction of the open position such that ambient air is suctioned into the housing and/or that the second housing part, starting from the open position, is movable in the direction of the closed position such that air enriched with the volatile substance is pressed out of the housing. Using successive opening and closing of the housing, e.g., successive transfer from the closed position to the open position and then back towards the closed position, it is possible to attain a brief stronger output of the volatile substance from the apparatus, for example, a brief stronger release of fragrance into the ambient air.

One alternative advantageous embodiment provides that the housing has a receiving element for the insert and one or a plurality of through-holes through which the volatile substance can be released into the ambient air, wherein the through-holes are provided with a removable barrier element, for example a film. The barrier element can be embodied, for example, as a release film. Alternatively, it can be provided that the removable barrier element is a removable housing element made of the first material of the housing. For example, the removable housing element can be connected to the rest of the housing via a perforation. This perforation can be separated when the housing element is removed.

Further details, features, and advantages of the invention result from the drawings and from the following description of preferred embodiments with the drawings. The drawings merely illustrate exemplary embodiments of the invention that do not limit the inventive thought.

### BRIEF DESCRIPTION OF THE FIGURES

FIG. 1 is a perspective elevation of an apparatus for releasing a volatile substance into the ambient air according to a first exemplary embodiment in a closed position.

FIG. 2 illustrates the apparatus according to FIG. 1 when the vapor barrier element is detached.

FIG. 3 illustrates the apparatus according to FIG. 1 in an open position.

FIG. 4 is a perspective elevation of an apparatus for releasing a volatile substance into the ambient air according to a second exemplary embodiment in a closed position.

FIG. 5 illustrates the apparatus according to FIG. 3 in an open position.

FIG. 6 is a perspective elevation of an apparatus for releasing a volatile substance into the ambient air according to a third exemplary embodiment in an open position.

FIG. 7 illustrates an inner housing part of a housing of the apparatus according to FIG. 6.

FIG. 8 illustrates the apparatus according to FIG. 6 in a closed position.

FIG. 9a is a top view of an insert of the apparatus according to FIG. 1.

FIG. 9b is a side view of an insert of the apparatus according to FIG. 1.

FIG. 10a is a top view of an insert of the apparatus according to FIG. 4.

FIG. 10b is a side view of an insert of the apparatus according to FIG. 4.

FIG. 11 is a perspective elevation of an alternative insert of the apparatus according to FIGS. 1, 4, and 6.

### DETAILED DESCRIPTION

In the various figures, identical parts are always provided with the same reference number and therefore as a rule each is also identified or mentioned only once.

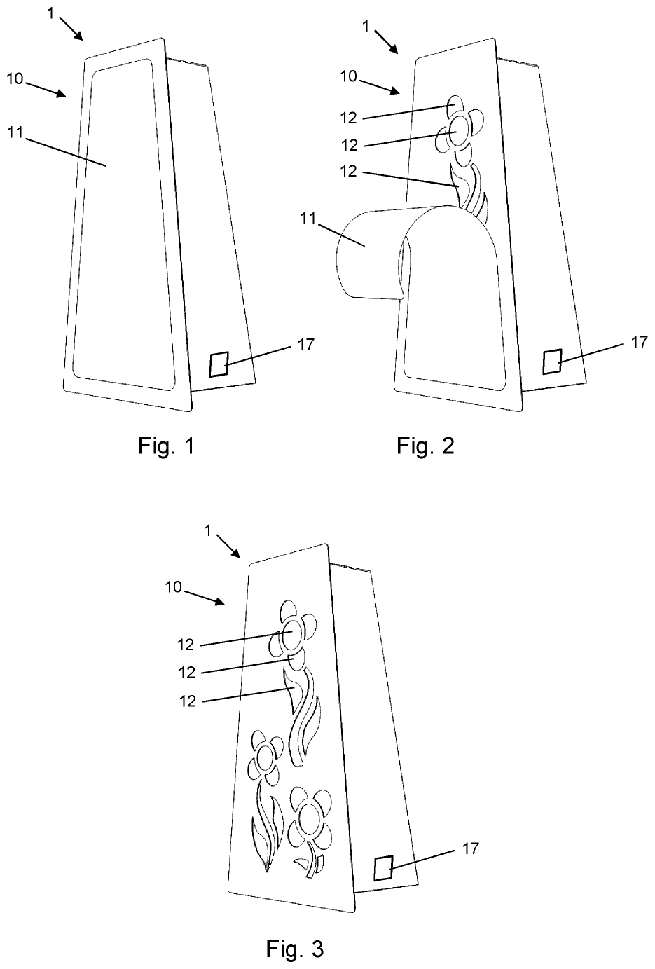

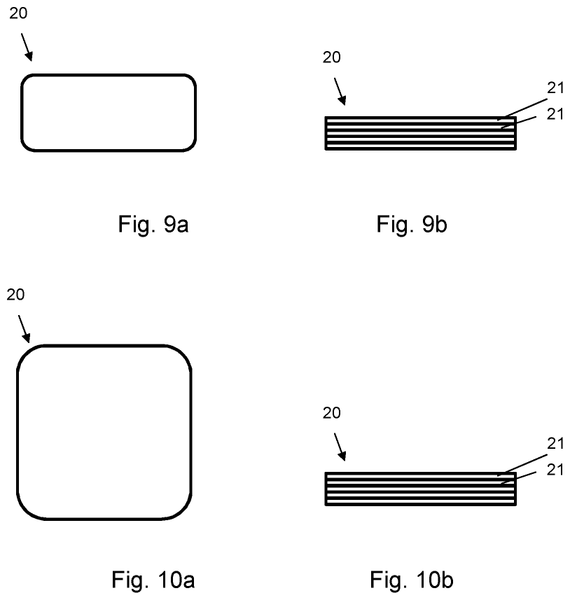

The depiction in FIG. 1 illustrates an apparatus 1 for releasing a volatile substance, for example, in the form of a fragrance or an insecticide, into the ambient air according to a first exemplary embodiment of the invention. The apparatus 1 comprises a housing 10 made of a first material having a plant-based fiber material, in particular paper, cardboard, or paperboard, and a barrier layer having a barrier function against permeation of the volatile substance as well as an insert 20, depicted in FIGS. 9a and 9b, arranged inside the housing 10. The insert 20 comprises a second material that is a plant-based fiber material, in particular paper, cardboard, or paperboard, wherein the second material is provided, in particular impregnated, with the volatile substance.

The housing 10 comprises a receiving element for the insert and one or a plurality of through-holes 12 through which the volatile substance can be released into the ambient air, wherein, in a closed position of the housing 10, the through-holes 12 are provided with a removable barrier element 11, for example a film, see FIG. 1. Alternatively, it can be provided that the removable barrier element 11 is a removable housing element made of the first material of the housing 10.

For example, the removable housing element can be connected to the rest of the housing 10 via a perforation. This perforation can be separated when the housing element 11 is removed.

The depiction in FIG. 2 depicts by way of example removal of this barrier element 11 from the through-holes 12.

In FIG. 3, the housing 10 is shown in an open position in which the through-holes are exposed so that ambient air can circulate through the latter and the volatile substance, for example a fragrance, of the insert 20 can exit from the interior of the housing 10. The barrier element 11 has been completely removed from the housing 10 according to FIG. 3.

The first material of the housing 10 of the apparatus 1 has a barrier layer, for example comprising polyamide, polyester, or polyvinyl alcohol. This barrier layer can be arranged in contact with a ply made of the plant-based fiber material. Furthermore, it can be provided that the barrier layer is embodied as a coating of a ply made of the plant-based fiber material, in particular as a coating on an inner side of the housing 10.

Furthermore, a time-temperature indicator 17 via which the shelf life of the volatile substance can be displayed is arranged on the outside of the housing 10.

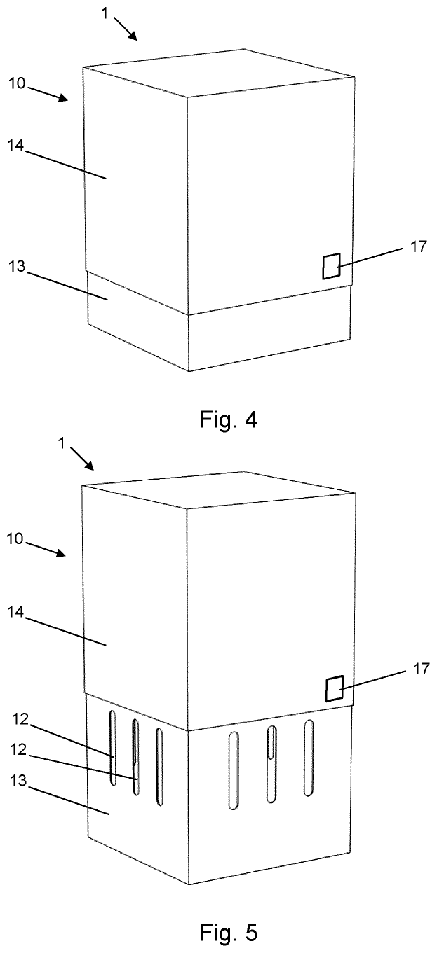

A second exemplary embodiment of an inventive apparatus 1 for releasing a volatile substance into the ambient air is depicted in FIGS. 4 and 5. The apparatus 1 according to the second exemplary embodiment also comprises a housing 10 made of a first material that has a plant-based fiber material, in particular paper, cardboard, or paperboard, and a barrier layer having a barrier function against permeation of the volatile material, as well as an insert 20, depicted in FIGS. 10a and 10b, arranged inside the housing 10. The insert 20 comprises a second material that is a plant-based fiber material, in particular paper, carton, or paperboard, wherein the second material is provided, in particular impregnated, with the volatile substance.

What has been stated about the first material in accordance with the first exemplary embodiment also applies to the second exemplary embodiment. Therefore refer to the description of FIGS. 1-3.

In contrast to the first exemplary embodiment, the housing 10 according to the second exemplary embodiment comprises a first housing part 13 having a recess for the insert, wherein the first housing part 13 has one or a plurality of through-holes 12 through which the volatile substance can be released into the ambient air, and a second housing part 14, which can be displaced out of a closed position, shown in FIG. 4, in which position the second housing part covers the through-holes 12 of the first housing part, into an open position, shown in FIG. 5, in which the through-holes of the first housing part are exposed for releasing the volatile substance.

The housing 10 according to the second exemplary embodiment also has a time-temperature indicator 17 via which the shelf-life of the fragrance or insecticide can be displayed.

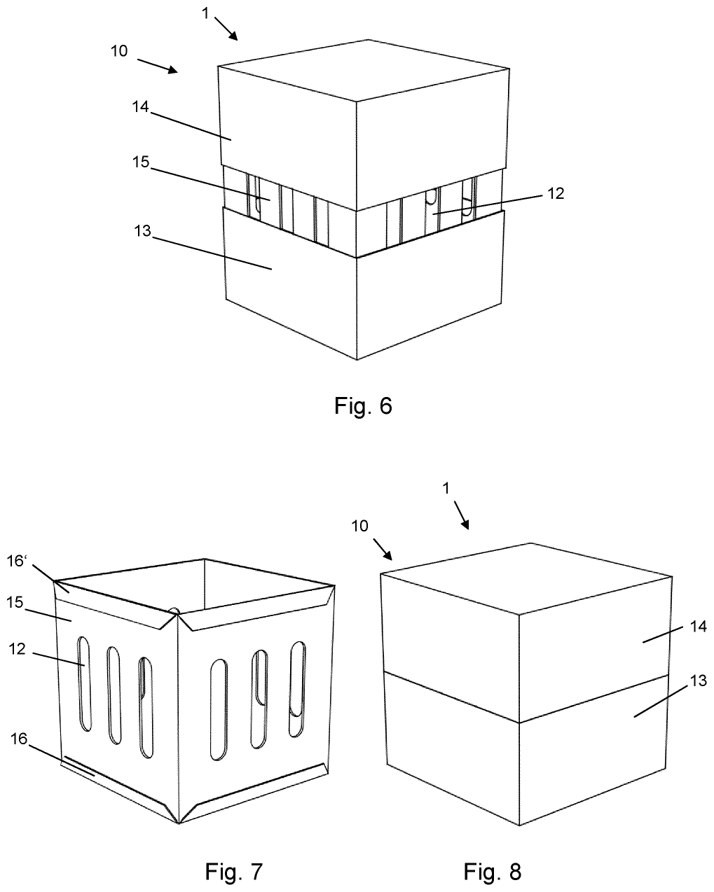

FIG. 6 depicts a third exemplary embodiment of an inventive apparatus 1 for releasing a volatile substance into the ambient air. The apparatus 1 according to the third exemplary embodiment also comprises a housing 10 made of a first material having a plant-based fiber material, in particular paper, cardboard, or paperboard, and a barrier layer having a barrier function against permeation of the volatile substance, as well as an insert 20, depicted in FIGS. 10a and 10b and arranged inside the housing 10. The insert 20 comprises a second material that is a plant-based fiber material, in particular paper, cardboard, or paperboard, wherein the second material is provided, in particular impregnated, with the volatile substance.

What has been stated about the first material in accordance with the first exemplary embodiment also applies to the second exemplary embodiment. Refer to the description of FIGS. 1-3.

The housing 10 of the apparatus 1 according to the third exemplary embodiment comprises a first housing part 13, a second housing part 14, and a third housing part 15. The first and second housing parts 13, 14 are embodied identically in the exemplary embodiment. Both the first and also the second housing part 13, 14 have a receiving element for the insert 20; only one of the two housing parts 13, 14 is fitted with the insert 20, however.

The third housing part 15 is embodied like a tube or sleeve and comprises a plurality of through-holes 12 through which the volatile substance can be released into the ambient air if they are exposed by the first and/or second housing part 13, 14. In the closed position shown in FIG. 8, the first housing part 13 and the second housing part 14 cover the through-holes 12 of the third housing part 15. The first and second housing parts 13, 14 can be displaced from this closed position into an open position, shown in FIG. 6, in which the through-holes 12 of the third housing part 15 are exposed for releasing the volatile substance.

The housing 10 can additionally have a removable barrier element, for example a barrier element embodied as a sleeve, that covers a gap between the first housing part 13 and the second housing part 14 in the closed position. This barrier element comprises a barrier material having a barrier function against permeation of the volatile substance or is embodied of such a material.

The third housing part 15 of the housing 10 is depicted in FIG. 7. It can be seen from this depiction that a plurality of first locking elements 16 are arranged on the third housing part and can cooperate with further first locking elements on the first housing part 13. Furthermore, the third housing part 15 comprises a plurality of second locking elements 16′ that can cooperate with further second locking elements on the second housing part 14. The first locking elements 16 are arranged on a first side of the third housing part 15 opposing a second side of the third housing part on which the second locking elements 16′ are arranged. Thus, when the housing is assembled, the first locking elements 16 face the first housing part 13 and the second locking elements 16′ face the second housing part. The first and second locking elements 16, 16′ are embodied as folded segments of the third housing part 13.

FIG. 8 depicts a housing 10 in the closed position having a first housing part 13 and a second housing part 14.

FIGS. 9a and 9b depict the insert 20 of the first exemplary embodiment. This insert 20 has a rectangular section with rounded corners. The insert 20 comprises a plurality of, in this case five, plys 21 of the plant-based fiber material, for example mechanical pulp board or wood pulp, connected to one another by an adhesive. The adhesive is preferably biodegradable. The plys each have an area density in the range of 350 g/m² to 950 g/m², preferably 450 g/m² to 600 g/m², or 600 g/m² to 800 g/m². Alternatively, it can be provided that the plys each have a thickness in the range of 0.8 mm to 2.5 mm and an area density in the range of 350 g/m² to 950 g/m².

FIGS. 10a and 10b depict the insert 20 of the second and third exemplary embodiments. This insert 20 has a square section with rounded corners. The insert 20 comprises a plurality of, in this case five, plys 21 of the plant-based fiber material, for example mechanical pulp board or wood pulp, connected to one another by an adhesive. The adhesive is preferably biodegradable. The plys each have an area density in the range of 350 g/m² to 950 g/m², preferably 450 g/m² to 600 g/m², or 600 g/m² to 800 g/m². Alternatively, it can be provided that the plys each have a thickness in the range of 0.8 mm to 2.5 mm and an area density in the range of 350 g/m² to 950 g/m².

The exemplary embodiments of the inserts 20 depicted in FIGS. 6a, 6b, 7a, and 7b are merely by way of example and can have a different shape, e.g. can be pentagonal, hexagonal, or octagonal, and/or can have sharp, non-rounded corners.

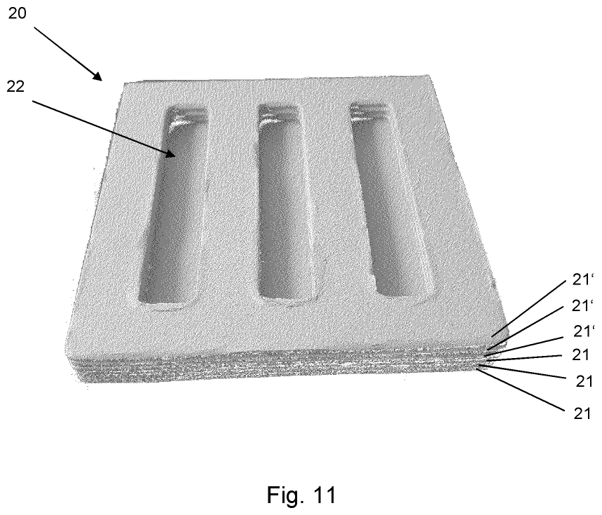

FIG. 11 depicts an insert 20 that can be used as alternative in the exemplary embodiments described in the foregoing. The insert 20 comprises a plurality of, in this case six, plys 21 of the plant-based fiber material, for example mechanical pulp board or wood pulp, connected to one another by an adhesive. The insert 20 as a plurality of, in this case three, first plys 21 that are embodied with the full surface area. The insert 20 furthermore has a plurality of, in this case three, second plys 21′ that include a plurality of, in this case three, in particular punched, holes. One of the second plys 21′ forms an outer surface of the insert 20. The recesses 22 of the second plys 21′ are arranged congruently above one another and form a receiving element for volatile material.

The inserts 20 described in the foregoing can alternatively comprise one of the following materials or one or a plurality of the cited materials: a further plant-based fiber material or sucrose or starch or a mineral material. The plant-based fiber material can be mechanical pulp, in particular ground wood pulp, or pulp or cotton fiber or grass fiber. The mineral material can be rock flour or bentonite or silica gel.

### LIST OF REFERENCE NUMBERS

1 Apparatus for releasing a fragrance or insecticide into the ambient air
10 Housing
11 Barrier element
12 Through-hole
13 First housing part
14 Second housing part
15 Third housing part
16, 16′ Locking element
17 Time-temperature indicator
20 Insert
21, 21′ Ply
22 Recess

1. Apparatus for releasing a volatile substance into ambient air, the apparatus comprising:
a housing made of a first material which has a plant-based fiber material, and a barrier layer having a barrier function against permeation of the volatile substance, and
arranged inside the housing, the apparatus comprises an insert made of a second material, the second material is provided or impregnated with the volatile substance, wherein the second material comprises one or more of:
i) plant-based fiber material or
ii) sucrose or starch or
iii) a mineral material.

2. Apparatus according to claim 1, wherein the barrier layer comprises polyamide, polyester, aluminum, or polyvinyl alcohol.

3. Apparatus according to claim 2, wherein the barrier layer is arranged in contact with a ply made of the plant-based fiber material.

4. Apparatus according to claim 1, wherein the barrier layer is arranged between two plys made of the plant-based fiber material.

5. Apparatus according to claim 2, wherein the barrier layer is embodied as a coating of a ply made of the plant based fiber material and/or as a coating on an inner side of the housing.

6. Apparatus according to claim 1, wherein the first material has a plurality of barrier layers that preferably have different permeabilities for different volatile substances.

7. Apparatus according to claim 1, wherein the housing has a stand surface on which the housing can stand on a subsurface, wherein the stand surface is water-repellant.

8. Apparatus according to claim 1, wherein the plant-based fiber material is paper or cardboard or paperboard, or mechanical pulp board, or wood pulp.

9. Apparatus according to claim 1, wherein the plant-based material is mechanical pulp, or ground wood pulp, or pulp or cotton fiber or grass fiber.

10. Apparatus according to claim 1, wherein the mineral material is rock flour or bentonite or silica gel.

11. Apparatus according to claim 1, wherein the insert has a plurality of plys of the second material connected to one another by an adhesive.

12. Apparatus according to claim 1, wherein the insert has a plurality of plys of mechanical pulp board connected to one another.

13. (canceled)

14. (canceled)

15. (canceled)

16. (canceled)

17. (canceled)

18. (canceled)

19. (canceled)

20. (canceled)

21. (canceled)

22. (canceled)

23. Apparatus according to claim 1, wherein the housing has a first housing part and a second housing part, the first housing part and/or the second housing part comprises a receiving element for the insert, wherein the first housing part comprises one or a plurality of through-holes through which the volatile substance can be released into the ambient air, and the second housing part can be displaced from a closed position, in which it covers the through-holes of the first housing part, to an open position, in which the through-holes of the first housing part are exposed for releasing the volatile substance.

24. Apparatus according to claim 23, wherein the first housing part and the second housing part have a basic outline of identical shape.

25. Apparatus according to claim 23, wherein a locking element that prevents complete separation of the first housing part and the second housing part from one another is arranged on the first housing part and/or second housing part.

26. Apparatus according to claim 1, wherein the housing has a first housing part, a second housing part, and a third housing part,
wherein the first housing part and/or the second housing part comprises a receiving element for the insert, wherein the third housing part has one or a plurality of through-holes through which the volatile substance can be released into the ambient air,
wherein the first housing part and/or the second housing part can be displaced from a closed position, in which the first housing part and/or the second housing part covers the through-holes of the third housing part, to an open position, in which the through-holes of the third housing part are exposed for releasing the volatile substance.

27. Apparatus according to claim 26, wherein the first housing part and the second housing part are embodied identical.

28. Apparatus according to claim 26, wherein the third housing part has the shape of a tube.

29. Apparatus according to claim 26, wherein a first locking element is arranged on the third housing part and cooperates with a further first locking element on the first housing part and a second locking element is arranged on the third housing part and cooperates with a further second locking element on the second housing part.

30. (canceled)

31. (canceled)

32. Apparatus according to claim 1, wherein the housing has a recess for the insert and one or a plurality of through-holes through which the volatile substance can be released into the ambient air, wherein the through-holes are provided with a removable barrier element, for and/or a film.
